(19) United States
(12) Patent Application Publication
(10) Pub. No.: US 2008/0262414 A1
(43) Pub. Date: Oct. 23, 2008

(54) SINGLE USE APPLICATOR CARTRIDGE FOR AN ELECTROKINETIC DELIVERY SYSTEM AND METHOD FOR SELF ADMINISTRATION OF MEDICAMENTS

(75) Inventors: Michael S. Barsness, Oxford, MA (US); Robert W. Etheredge, Natick, MA (US)

Correspondence Address: NIXON & VANDERHYE, PC, 901 NORTH GLEBE ROAD, 11TH FLOOR, ARLINGTON, VA, 22203, US

(73) Assignee: TRANSPORT PHARMACEUTICALS, INC., Framingham, MA (US)

(21) Appl. No.: 12/105,764

(22) Filed: Apr. 18, 2008

Related U.S. Application Data
(60) Provisional application No. 60/913,151, filed on Apr. 20, 2007.

(51) Int. Cl. A61N 1/30

(52) U.S. Cl. 604/20

(57) ABSTRACT

An applicator cartridge for use with a device for electrokinetically delivering a medicament to a treatment site, the cartridge including: an applicator head including an active electrode, and a conductive path extending between a pair of electrical contacts, wherein the electrical contacts electrically engage respective contacts on the device when the cartridge is inserted into the device; a matrix support surface of said applicator head adjacent the active electrode; a matrix attached to the matrix support surface and in contact with the active electrode, the matrix having an exposed surface adapted to be applied to the treatment site; a medicament or a medicament and an electrically conductive carrier carried by said matrix; a removable lid covering the exposed surface of the matrix and sealed to the applicator head, the lid is conductively coupled to the applicator head and completes the conductive path on the applicator head, wherein removal of the lid breaks the conductive path.

### RELATED APPLICATION

This application claims the benefit of U.S. Provisional Patent Application Ser. No. 60/913,151 filed Apr. 20, 2007, the entirety of which is incorporated by reference.

### BACKGROUND OF INVENTION

The present invention relates generally to applicators for electrokinetic mass transfer of substances to live tissue and particularly relates to an apparatus for electrokinetically delivering substances, e.g., a medicament, to a treatment site on skin.

Electrokinetic delivery of medicaments for applying medication locally through a human individual's skin to a treatment site is known. One type of electrokinetic delivery mechanism is iontophoresis, i.e., the application of an electric field to the skin to enhance the skin's permeability and to deliver various ionic agents, e.g., ions of salts or other drugs to the treatment site. In certain situations, iontophoretic or transdermal or transmucosal cutaneous delivery techniques have obviated the need for hypodermic injection of many medicaments thereby eliminating the concomitant problem of trauma, pain and risk of infection to the individual. Other types of electrokinetic delivery mechanisms include electroosmosis, electroporation, and electromigration, any or all of which are more generally known as electrotransport, electromolecular transport or iontophoretic methods, all of which are collectively known as electrokinetic methods.

Electrokinetic devices have been developed for the private self administration of medicaments or for diagnostic application by the individual at non-medical or non-professional facilities. For example, U.S. Pat. No. 6,792,306 and U.S. Published Patent Application No. 2006/0167403, disclose electrokinetic delivery devices which include a housing containing a power source, electronics and a counter electrode, the device being shaped and configured for releasable securement to an individual's finger and terminating in an applicator head having an active electrode. By applying the applicator head to the skin overlying the treatment site and with the medicament or a medicament and a carrier therefor carried by the applicator head, the medicament may be electrokinetically delivered to the treatment site.

The applicator head of the electrokinetic device is typically releasable. The head may include a cartridge containing a medicament matrix, active electrode and connecting prongs that fit into a receiver of the device. The user inserts the cartridge into the receiver of the device, removes a lid from the face of the cartridge to expose the medicament matrix, applies the matrix and front of the cartridge to the treatment site, and activates the device to deliver the medicine through the site. After delivery of the medicine (medicament) the user removes the cartridge from the device, and reinserts another cartridge for a subsequent application of medicament to another user, to another treatment site, or to the same treatment site at a later application time.

The cartridge may be intended by its manufacturer to be a single use cartridge to be discarded or returned to the manufacturer after a single use. Once the medicament has been discharged from the cartridge, the cartridge is no longer suitable for delivering medicine. Once used, the cartridge is to be removed from the electrokinetic device and not later inserted into the device.

There is a risk that a used cartridge will be inadvertently inserted into the device and the user may mistakenly attempt to deliver medicine to the treatment site with the used cartridge. Further, an unscrupulous third-party vendor may intentionally sell a used cartridge, which may or may not have a new medicament pad with effective medicine, to users who believe that they are purchasing a valid and safe medicament cartridge. The user may unknowingly insert the used cartridge in the electrokinetic device believing that he is about to apply medicament to a treatment site. The medicament may no longer be present in the medicament pad in an effective amount or the medicament in the pad may be an incorrect amount, an improper medicament or have some other defect. These dangers of reusing a cartridge create a need for mechanisms and methods to prevent reuse of a cartridge.

There has existed an increasing imperative to develop a safety mechanism and method that prevent reuse of a medicament cartridge in an electrokinetic device. Preferably, the safety mechanism should reliably detect the insertion of a used cartridge into an electrokinetic device and disable the device when the used cartridge is inserted. In addition, it would be advantageous for the safety mechanism to be readily and easily manufactured at low cost, not affect the normal operation of a new cartridge, and not interfere with the delivery of medicament from a new cartridge.

### SUMMARY OF INVENTION

An applicator cartridge for use with a device for electrokinetically delivering a medicament to a treatment site, the cartridge comprising: an applicator head including an active electrode, and a conductive path extending between a pair of electrical contacts, wherein the electrical contacts electrically engage respective contacts on the device when the cartridge is inserted into the device; a matrix support surface of said applicator head adjacent the active electrode; a matrix attached to the matrix support surface and in contact with the active electrode, the matrix having an exposed surface adapted to be applied to the treatment site; a medicament or a medicament and an electrically conductive carrier carried by said matrix; a removable lid covering the exposed surface of the matrix and sealed to the applicator head, the lid is conductively coupled to the applicator head and completes the conductive path on the applicator head, wherein removal of the lid breaks the conductive path.

An applicator cartridge for use with a device for electrokinetically delivering a medicament to a treatment site, the cartridge having: an applicator head including an active electrode, and a conductive path extending between a pair of electrical contacts, wherein the electrical contacts electrically engage respective contacts on the device when the cartridge is inserted into the device; a matrix support surface of said applicator head adjacent the active electrode; a matrix attached to the matrix support surface and in contact with the active electrode, the matrix having an exposed surface adapted to be applied to the treatment site; a medicament or a medicament and an electrically conductive carrier carried by said matrix; and a removable lid covering the exposed surface of the matrix and sealed to the applicator head, the lid including a conductive contact sealed to the applicator head and completing the conductive path on the applicator head, wherein removal of the lid breaks the conductive path.

An applicator cartridge for use with a device for electrokinetically delivering a medicament to a treatment site comprising: an applicator head including an active electrode and a conductive path extending between contacts on the head wherein the contacts electrically contact the device when the cartridge is inserted in the device; a matrix support surface of said applicator head adjacent the active electrode; a matrix attached to the matrix support surface and in contact with the active electrode, the matrix having an exposed surface adapted to be applied to the treatment site; a medicament or a medicament and an electrically conductive carrier carried by said matrix; a removable lid covering the exposed surface of the matrix and sealed to the applicator head, the lid including a tab sealed to the applicator head and engaging the conductive path extending between contacts on the applicator head, wherein removal of the lid breaks the conductive path.

A method has been developed to prevent reuse of an applicator cartridge for use with a device for electrokinetically delivering medicament to a treatment site on skin, the method comprising: mounting a medicament pad in an applicator head wherein the medicament pad is in electrical contact with an active electrode and has an exposed front surface to be applied to the treatment site; sealing a lid to the applicator cartridge to cover the front surface of the medicament pad; establishing a conductive path on the applicator head that extends between contacts on the head; inserting the applicator cartridge in the device and making electrical connections between the device and the contacts on the cartridge; applying an electrical current from the device through the conductive path on the cartridge; if the electrical current passes through the conductive path, authorizing the device to deliver the medicament to the treatment site; removing the lid from the applicator cartridge; applying the exposed front surface to the treatment site and electrokinetically delivering the medicament through the treatment site, and by the removal of the lid, breaking the conductive path and disabling the device from delivering the medicament to the treatment site if the cartridge is reinserted into the device.

A method has been developed for preventing reuse of an applicator cartridge for use with a device for electrokinetically delivering medicament to a treatment site on skin, the method comprising: mounting a medicament pad in the applicator cartridge wherein the medicament pad is in electrical contact with an active electrode and has an exposed front surface to be applied to the treatment site; including an electronically readable indicia on the applicator cartridge, wherein the indicia identifies the cartridge; inserting the applicator cartridge in the device and making electrical connections between the device and the active electrode and with the indicia; electronically reading the indicia with an processor or logic circuit in device to determine one or more of whether the cartridge is a previously unused cartridge, the type of the cartridge, and the medicament in the cartridge, and based on the determination, the processor or logic circuit applying zero or a predetermined amount of electrical current to the active electrode and thereby electrokinetically delivering the medicament to the treatment site.

### DESCRIPTION OF THE DRAWINGS

FIG. 1 is a perspective view of an exemplary electrokinetic delivery device including an applicator cartridge head secured to the delivery device.

FIG. 2 is a view similar to FIG. 1 illustrating the device with one side of the housing removed to show internal components.

FIG. 3 is an exploded view of the applicator cartridge as viewed from its backside.

FIG. 4 is an exploded view of the front of the applicator cartridge illustrating an applicator lid, matrix and cartridge head as viewed from the front side of the cartridge, i.e., the side contacting the individual's skin.

FIG. 5 is a side perspective view of the applicator cartridge.

FIG. 6 is an enlarged cross-sectional view of the applicator cartridge secured to the device.

FIG. 7 is an enlarged cross-sectional view of a lid for the applicator head.

FIG. 8 is an exploded view of an applicator cartridge as viewed from its backside, wherein the applicator cartridge includes an alternative lid and electrical contact between the lid and cartridge.

FIG. 9 is a front view of electrical contact pads for the cartridge and device.

### DETAILED DESCRIPTION OF THE INVENTION

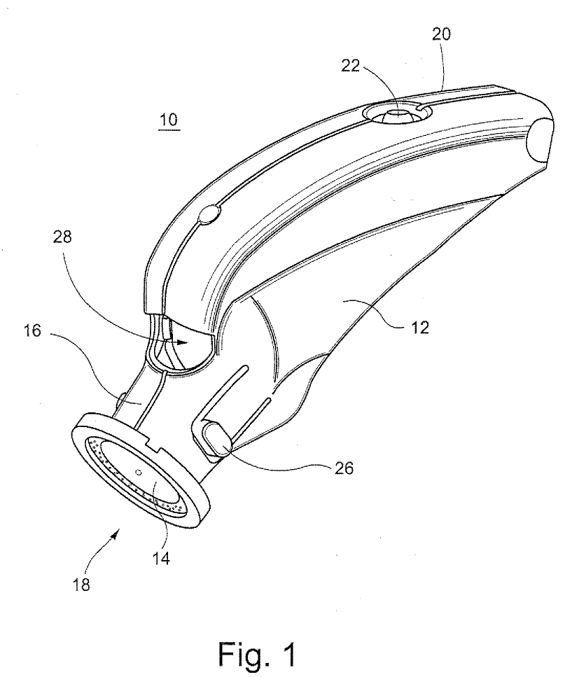

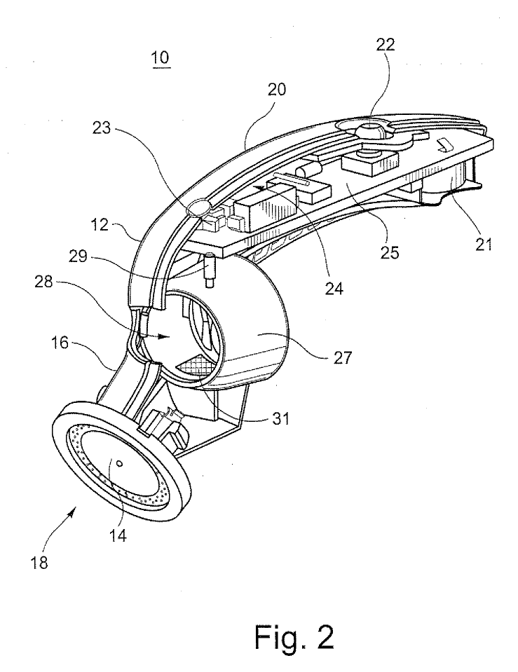

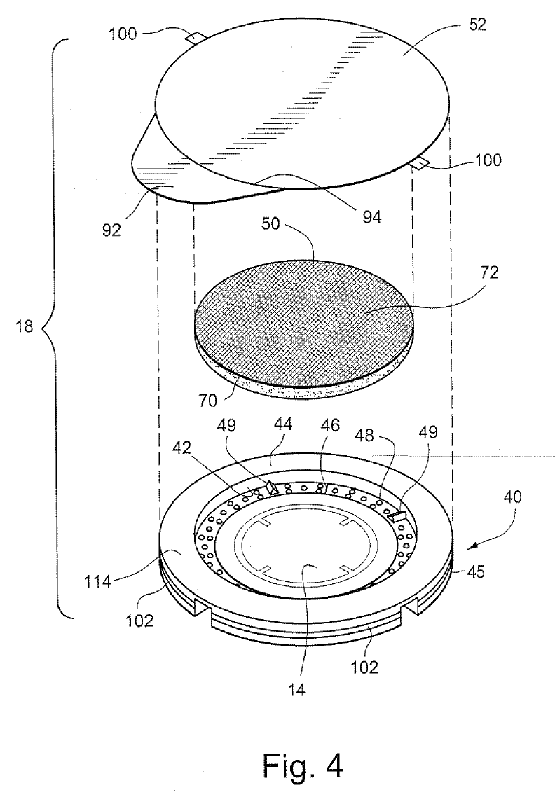

FIGS. 1 and 2 show a portable, self contained, lightweight, compact, finger mounted, electrokinetic medicament delivery device or medicator 10 (collectively a “device”) for application to a treatment site (TS) on the skin of an individual. The device 10 includes a housing 12 mountable to an individual's finger with the receiver 16 of the device 10 mounting an applicator cartridge 18 containing an active electrode 14. The device 10 drives, e.g., electrokinetically transports, medicament interposed between the active electrode 14 and the individual's treatment site into the treatment site upon completion of an electrical circuit through the device 10, the active electrode 14, the medicament or hydration material carrying the medicament (collectively referred to as “medicament”), the individual's body and a counter electrode 31, i.e., tactile electrode carried by the device. While FIG. 1 shows the active electrode 14 exposed on the front face of the cartridge 18, the electrode is typically covered by a medicament matrix (50 in FIG. 4) that is attached to a front face of the cartridge head and applied to the treatment site. The device 10 carrying the applicator cartridge 18 provides a facile and fatigue-free approach to the affected treatment site. The housing, in one embodiment, need not be secured to the finger with straps or other fixtures for immobilizing the finger.

The housing 12 may include an internal compartment 24 for receiving a printed circuit board 25 containing a battery power source 21 and possibly a micro controller. The printed circuit board 25 or other electronic package may control current applied to the active electrode, time of current delivery to the active electrode, provide redundant safety features (such as a switch to prevent application of the current without a cartridge head in the device), and ensure user visual and/ or audible signaling during use of the device, e.g., activation of the circuit board LED transmitted through the light pipe 23. A contact pin 29 provides electrical contact(s) between the cartridge and the printed circuit board. The pin may include multiple conductive paths.

A proximal portion 20 of the housing 12 is elongated and shaped to fit comfortably on the top of the user's index finger. Located on the top surface of the housing is a manually actuated switch button 22 for energizing the circuitry and preparing the device for use. An opening 28 in the housing that provides an access port through which long fingernails may extend. The opening allows finger nails and long fingers to project through the housing so that good contact may be made between the fleshy pad of the finger and the ring 27. The opening facilitates proper contact with the counter electrode, e.g., contact surface 31, on an inside surface of the ring 27.

Adjacent to either side of the fingernail port 28 are ejector buttons 26 that, when depressed, disengage the applicator cartridge 18 and provide a forward movement of the applicator cartridge away from the device during disposal. The exact size and position of the ejection and/or release features may be varied in response to the size of the applicator cartridge. The ejection and/or release features work in concert with the applicator and prevent inadvertent ejection or release during use.

The applicator cartridge 18 is preferably secured to a receiver 16 in a the distal end of the device 10. The receiver 16 includes a releasable coupling, e.g., prongs 62, that engage inside surfaces of a hollow receiver section of the receiver 16. By providing a two part device 10, e.g., housing 12 and cartridge 18, a fresh applicator cartridge 18 can be applied to the housing 12 for each subsequent use of the device 10. The used applicator cartridge 18 can be ejected or released from the housing by a one-hand operation. A new applicator cartridge head is attached to the device for subsequent treatments.

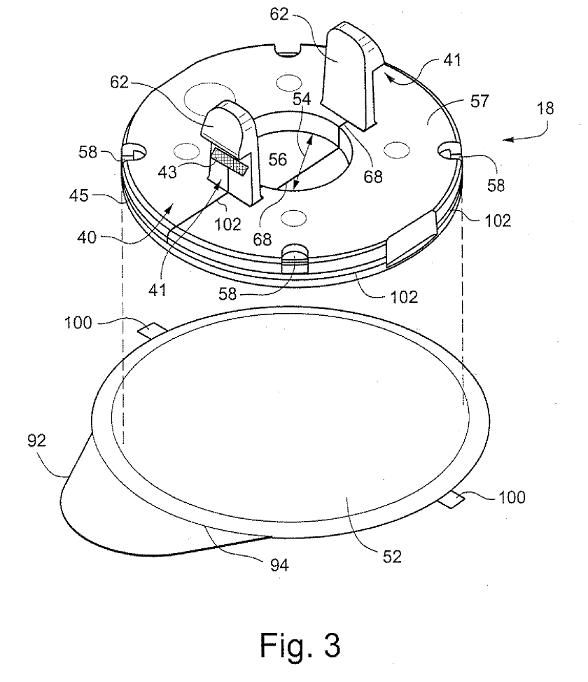

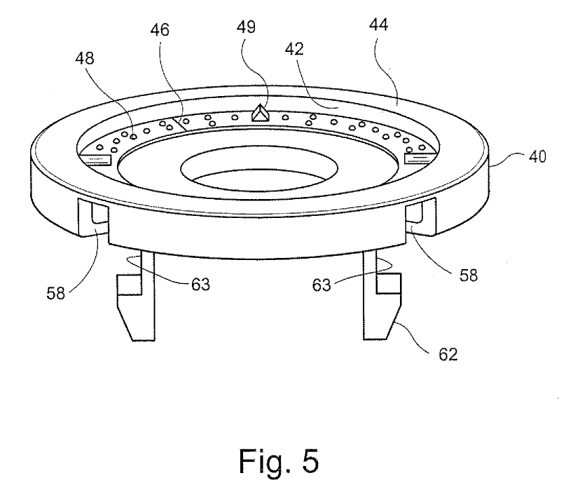

Referring to FIGS. 3 to 6, the applicator 18 generally includes an applicator cartridge head 40, e.g., a disc, having on its back or rear side a locking element 41, e.g., prongs 62, for securing the cartridge 18 to the device 10. The head 40 of the applicator cartridge 18 includes a circular recess 42 on a forward face 114 of the head and defined by a rim 44 of the head 40. An active electrode surface 14 is disposed within the recess 42. The electrode surface may be a separate metal part, a metallized coating on one or more regions of the recess, a conductive polymer attached to the applicator head, or other means for providing electrical contact between the power source and the cartridge recess.

A flexible lid 52 having a generally disc shape with tabs 92 and 100. The lid is sealed to the rim 44 of the front face 114 of the cartridge head 40. The lid when sealed to the rim shields the medicament matrix 50 contained in the cartridge. The lid 52 safeguards the medicament matrix from tampering, damage and other harm. The sealed medicament matrix 50 is protected by the lid until the lid is removed just prior to dispensing of the medicament at the treatment site. Premature removal of the lid, such as before the cartridge is inserted into the device, may expose the medicament matrix to tampering, damage or other harm. Premature removal of the lid poses risks to patient health and potential degradation of effectiveness of the medicament.

The lid 52 includes at least one tab 100 that extends outward from the circumference of the disc portion of the lid. When the lid is sealed to the rim 44 of the cartridge, the tab(s) 100 make electrical contact with the electrode 14 through a foil strip 102 located on side wall 45 of the cartridge 18. The conductive path extends across the lid and from either side of the lid through tabs 100 to one or more contact pads 43 on the connecting prongs 62 of the cartridge. The tab(s) 100 may attach to the cartridge 18 such that when the lid 52 is removed from the cartridge the conductive path is broken such that a current does not flow. The conductive path 102 thus comprises, for example, the contact pads 43, the conductive foil strip on the backside of the cartridge and on the sidewall 45 of the cartridge head 40, and the lid 52. The foil strip forming the conductive path 102 may be releasably attached to the sidewall 45 in a region of the tabs 100. The tabs 100 may be fixed, e.g., bonded, to the foil strip such that the removal of the lid and tabs breaks the foil strip and interrupts the conductivity of the conductive paths 102.

The contact pads 43 electrically engage contact pads 65 on the device, when the cartridge prongs 62 are inserted in the receiver 16 at the distal end of the device. For example, the contact pads 43 engage opposing contacts 65 on an inner wall 63 of the distal portion of the device. The contacts 65 on the inner wall 63 of the device are electrically coupled to the printed circuit board 25 and power supply 21, which, for example, apply an electrical potential across contacts on opposite sides of the inner wall 63 and to the contact pads 43 that engage the contacts when the cartridge is inserted into the receiver of the device.

The recessed base 42 within the rim 44 of the applicator may be shaped to create a concave recess or other profile complementary to the shapes of the electrode and matrix. In a preferred embodiment the perimeter of the active electrode 14 does not extend to the inner wall of the rim 44. An annular surface 46 of the recess remains between the rim and active electrode. A multitude of electrode sizes, shapes, and materials may be used to provide electrical contact between the applicator recess and the matrix. Also in the preferred embodiment the annular surface 46 includes a plurality of raised projections, e.g., raised dimples 48, ridges 49, or combinations thereof of variable heights projecting from the surface 46. The dimples 48 may be radially spaced from each other across the surface 46.

The active electrode 14 may be composed of metal, a metallized polymer or a conductive polymer such as polyaniline, polypyrrole, or a polymer rendered conductive by means of a conductive dopant. The removable lid 52 over the front face of the head 40 is composed of a polymer laminate with or without a metallic layer. The head 40 may be formed of a polymeric material, such as polypropylene or other polymer inert to the drug formulation in the matrix 50.

To provide an electrical connection between the active electrode 14 of the applicator 18 and the power source, the opposite or second face, e.g., the backside 57, of the head 40, has an opening 54, preferably central to the head 40 through which the backside of the active electrode 14 is exposed. An electrical connection is provided between the backside portion 56 of the electrode 14 and the circuit board when the applicator 18 is secured to the device. The backside 57 of applicator cartridge head 40 also includes one or more openings 58 which also expose a portion of the active electrode 14. The additional exposure of the electrode 14 facilitates the transmission of electrical signals for diagnostic testing during manufacture of the applicator 18 and provide a conductive connection to the lid tabs 100.

The backside 57 of applicator cartridge head 40 includes a pair of prongs 62 that form one part of the locking element 41. The receiver 16 of device 10 mounts a pair of flats 64 along an inner surface of the receiver 16. The flats are a second part of the locking element 41. By inserting the prongs within the open inner surface of receiver 16, the prong heads engage the device flats to secure the applicator 18 to the device.

The active electrode 14 makes electrical contact with the circuit board within the distal portion 16. The outer surface of the prongs 62 and an inner cylindrical wall 63 of the receiver 16 may be both electroplated to include conductive pads 43 on outer surface of the prongs and a conductive pad 65 on the inner wall 63 of the receiver, e.g., the hollow receiver 16 of the device. A conductive line 68 on the surfaces of the head 40 provide an electrical path between the conductive pad 43 on the prongs 62 and the back contact surface 56 of the active electrode. The conductive line 68 may be a bus that includes multiple conductive paths to transmit various signals from different contact points on the active electrode. The contact pads 43 may be segmented to provide electrically separate contacts for the conductive path 102 and for the active electrode.

When the prongs 62 are inserted in the distal portion 16, the electroplated surfaces 62, 63 abut to provide an electrical contact between the circuit board 25 in the housing and the active electrode 14. Further, the surface(s) of the prongs may carry indicia or other markings, e.g., on contact 42, for lot traceability, medicament identification, prevention of reuse of the applicator, cartridge tamper protection and other information that are “read” by the microprocessor or other logic circuit controlled circuitry in the device.

The matrix 50 is a carrier supporting the medicament. Acceptable materials for the matrix include but are not limited to variable loft nonwoven and woven materials such as melt-blown, needlepunched, spunbonded, spunlaced or other processed natural fibers, polyolefin, polyester, rayon, nylon, and blends of these, reticulated polyether and polyester polyurethane foams, and silicone foams. Low void volume materials may also be used such as crosslinked hydrogels, phase change polymers, interpenetrating polymer networks, scaffolds for immobilizing the active prior to iontophoretic release, highly viscosified formulations, and other matrices that do not rely upon a delivery from a liquid formulation. The matrix may also contain functional components or layers 70 such as reinforcing scrims, networks, and other support structures to facilitate manufacture of the finished product. These layers may also be conductive to ensure homogeneous electrical contact with the drug formulation contained in the matrix. Additionally, the matrix may contain one or more layers carrying arrays of microneedles 72 or other surface features designed to physically penetrate the stratum corneum and promote delivery of medicaments intradermally or transdermally.

The porous matrix 50 may be a porous pad, membrane or substrate for the medicament. Acceptable materials for the porous matrix may include but are not limited to variable loft nonwoven and woven materials such as melt-blown, needlepunched, spunbonded, spunlaced or other processed polyolefin, polyester, rayon, nylon, and blends of these, reticulated polyether and polyester polyurethane foams, and silicone foams. Portions of the porous matrix may be conductive to ensure homogeneous electrical contact. The medicament or a medicament and hydration carrier for the medicament is disposed in the matrix 50, such as a non-woven material layer 70. The contact with the active electrode 14 is between the electrode 14 and the medicament or the medicament and its carrier.

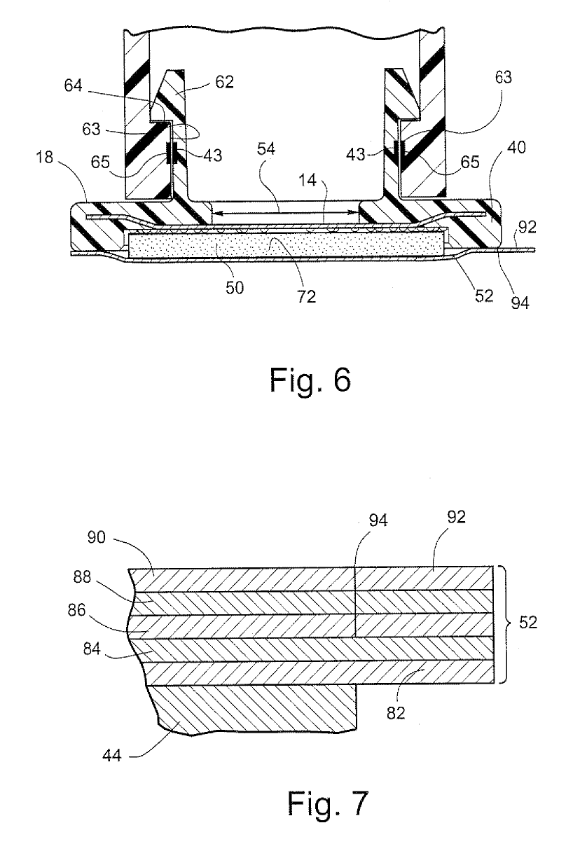

To maintain the applicator cartridge 18 in a sealed condition prior to use, the lid 52 is sealed to the outer rim 44 of the head 40. The lid 52 is formed of a plurality of layers. For example, as illustrated in FIG. 6, the bottom layer is a heat seal layer 82 followed by a plastic backing or polypropylene layer 84. A vapor barrier layer 86 lies intermediate the opposite faces of the lid followed by a foam layer 88 and a top polyester (Mylar®) layer 90. The vapor barrier layer may be comprised of metal foil, metallized polymer, or coating that prevents escape of volatiles from the applicator when the lid is sealed. In general, the lid 52 may be composed of any material providing a hermetic seal over the drug formulation and pad. The lid 52 additionally has a peel off tab 92 which preferably projects laterally from one side of the lid and beyond the rim 44 of the head. The peel off tab 92 may be one or more projections from the otherwise circular lid each of which are sufficiently large to be grasped by fingers of a user who is removing the lid. Alternatively, the tab may be a ring of the lid that projects laterally beyond the head 40 of the applicator. The tab 92 and the lid 52 are formed of the same layered material. The lid is preferably induction sealed about the outer rim 44 of the front face of the cartridge head 40. Induction sealing uses an RF field to create heat in a metallic layer, i.e., the middle layer 86 which in turn melts the polymer layer to effect a heat seal with the head 40 of the applicator cartridge 18. To prevent the tab 92 from acting as a heat sink which would cause the seal in the vicinity of the tab to lag behind the heat sealing of the lid to the head 40 in areas of the head remote from the tab, a discontinuity is provided between or at the interface of the tab and the lid. The discontinuity may be in the form of a kiss cut 94 which interrupts the thermal path to the tab from the lid when heat is applied. At the interface, at least the foil layer is cut, for example not less than 50-70% of its length along the tab/lid interface to prevent the tab from acting as a heat sink. If a tab formed of plastic or other insulator is used, there may be no need for a foil cut. The kiss cut 94 may not be necessary if the lid is secured to the head 40 by means other than induction heating, e.g., an adhesive or heat sealing. Also, the tab 92 need not project laterally as illustrated.

For example, a central pull tab, e.g., in a semi-circular form, may be used to remove the lid from the head 40. To utilize the electrokinetic delivery device 10, a cartridge head 40 is inserted into the receiver at the receiver 16 of the device. As the prongs 62 of the cartridge head engage the inner wall 63 of the receiver, the cartridge head is locked into the device 10. The conductive pads 43 on the prongs establish an electrical connection with opposing conductive pads 65 on the inner wall 63 of the receiver. The device, e.g., a microcontroller in the printed circuit board 25, monitors the conductivity between the opposing conductive pads 65 to sense whether a new cartridge head 40 is properly inserted into the receiver at the distal end and prepares to detect skin contact and to begin current ramp-up for therapy.

If and when the circuitry on the printed circuit board 25 determines that there is conductivity between opposing conductive pads 65 and/or if indicia on the prongs of the cartridge indicate that an unused cartridge has been inserted into the receiver, the device determines that a new cartridge is properly inserted in the device. The determination of whether a new cartridge is properly inserted may be based on either or both conductivity through the removable lid on the cartridge and an encoded electronically readable indicia marking the cartridge.

The removable lid on the cartridge may be optionally used by the device to determine if the cartridge is new and unused. The cartridge may include a tab(s) 100 that is applied to a conductive path 102 on the cartridge. Conductivity is present only if the conductive path 102 through the cartridge is not broken and electrically connects the opposition conductive pads 43 on the prongs of the cartridge. The conductive path 102 on the cartridge head may extend over the tab(s) 100 of the lid 52. When the cartridge is inserted into the device, the contacts 65 on the inner wall of the receiver of the device electrically engage the contacts 43 on the prongs of the cartridge. Current may flow from one of the pair of connected contacts 43, 65, through the conductive path 102, the opposite pair of connected contacts 43, 65 and to the

Once conductivity is determined, the device, e.g., a microcontroller on the printed circuit board, activates the circuitry for applying electrical current to the active electrode and the device. As discussed above, the circuitry on the printed circuit board reads a unique indicia, e.g., a number, printed on and identifying each cartridge and correlates this indicia to successful ramp-up of the current for therapy or other event denoting use of the cartridge. The circuitry may store in memory the indicia of a cartridge and store an indication that the cartridge corresponding to the indicia has been used. The circuitry may prevent usage of a cartridge that has previously be used. In this manner, the circuitry ensures that each cartridge is used only once.

The microcontroller may activate the circuitry in the device for a single application of medicament, e.g., a single treatment cycle of current applied to the active electrode. The microcontroller may in addition or alternatively activate the device for a predetermined period of time, e.g., 5 to 30 seconds or 1 to 15 minutes. By activating the device for a single application or a predetermined period of time, the microcontroller ensures that the cartridge head 40 in the device may be used for a single use. The composition of a single use may dictate how the microcontroller limits the activation of the device. For example, if a single use is a single application of a current regime, e.g., application of a constant current for 1 to 15 seconds, to the medicament matrix, the microcontroller may be programmed to activate the device for a single use. In another example, if a single use of a cartridge may include multiple actuations of the switch button 22 of the device (such as to apply the medicament pad to a plurality of treatment sites during a single treatment cycle), the microcontroller may active the device for a predetermined period.

Once the device is activated, the user removes the lid 52 from the cartridge housing to expose the front surface of the matrix 50. The user may then apply a conductivity enhancer (towelette, hydrogel, or equivalent) to the index finger, insert the finger into the opening 28 of the housing 12, and apply the front face of the medicament matrix and cartridge head to the skin at the treatment site. The device may be switched on, e.g., actuated, by the user depressing switch 22. When actuated, the printed circuit board applies a current to the active electrode and the current delivers medicament from the matrix 50 to the treatment site below the skin. The electrokinetic current flows from the device active electrode 14 through the face thus delivering the active into the skin and returns through the finger and into the counter electrode ring 60.

The removal of the lid breaks the conductive path 100 on the cartridge head 40. If the cartridge is later reinserted into the receiver of the housing 12, the microcontroller will not detect a continuity between the contact pads 65 and will not reactivate the device if the cartridge is reinserted into the receiver of the housing 12. Similarly, if the lid is prematurely removed before the cartridge is inserted into the housing 12, the microcontroller will not detect a continuity and will not activate the device. However, if the cartridge is marked with an indicia identifying the cartridge, the microprocessor may determine if the cartridge has been previously been used by comparing information read from the indicia to information stored in the memory of the microprocessor regarding previously used cartridges. If the comparison indicates that the cartridge has not been used, the microprocessor may authorize the application of current to the active electrode and thus delivery of medicament to the treatment site—even though there the conductive path was broken prior to insertion of the cartridge into the device. Having the microprocessor authorize the delivery of medicament based on a reading of the indicia on a cartridge allows a cartridge to be used, even if a user inadvertently removed the lid prior to placing the cartridge into the device.

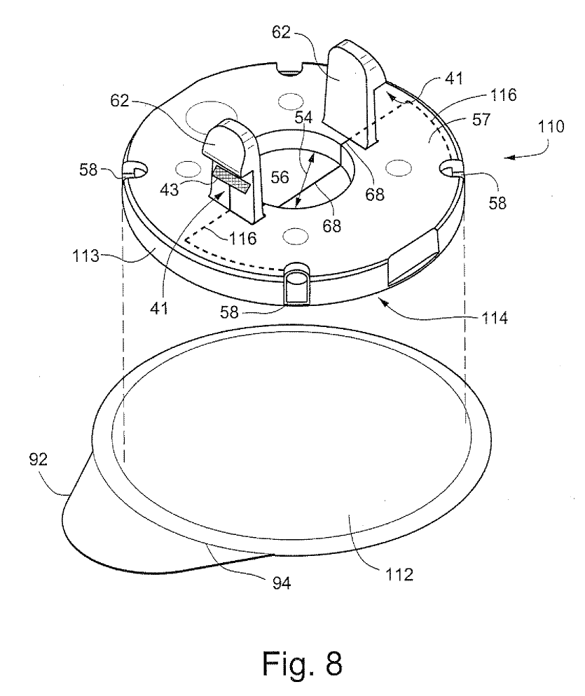

FIG. 8 is an exploded view of an applicator cartridge 110 as viewed from its backside, wherein the applicator cartridge includes an alternative lid 112 and electrical contact between the lid and cartridge. The lid 110 is electrically coupled to electrodes 58, e.g., metallic contact pins or foil strips, on or embedded in the sidewall 113 or front face 114 (hidden from view in FIG. 8 but shown in FIG. 4) of the cartridge head. The electrodes 58 may be on opposite sides of the cartridge head, but need only be spaced apart and both in contact with the lid. Further, the lid need not have a contact tab, especially if the electrodes 58 extend to the front face 114 (FIG. 4) that is in direct contact with the lid. However, the lid may include a tab(s) to provide electrical contact between the device, especially if the contact electrodes do not extend to the front face of the cartridge head 110.

When the lid is sealed to the cartridge, the lid 112 makes electrical contact with two (or more) of the electrodes 58 in the cartridge head 110. A conductive electrical path is formed from the contact pads 43 on the pair of prongs 62, through the conductive lines 116 that extends between the pads 43 and the electrodes 58. The conductive lines 116 are shown as dotted lines 116 to illustrate that the line may be on or below the backside 57 of the cartridge head. The conductive electrical path extends between the electrodes 58 and through the lid 112. The contact pads 43 connect the conductive path in the cartridge head 110 to the electronics and power supply of the device.

When the lid 112 is removed from the cartridge the conductive path is broken such that a current does not flow through the conductive path between the contact pads 43. If the lid is removed before cartridge insertion in the device or is not on the cartridge when the cartridge is inserted, the break in the conductive path is sensed by the device and the device may disable activation of the cartridge and dispensing of medicament from the cartridge.

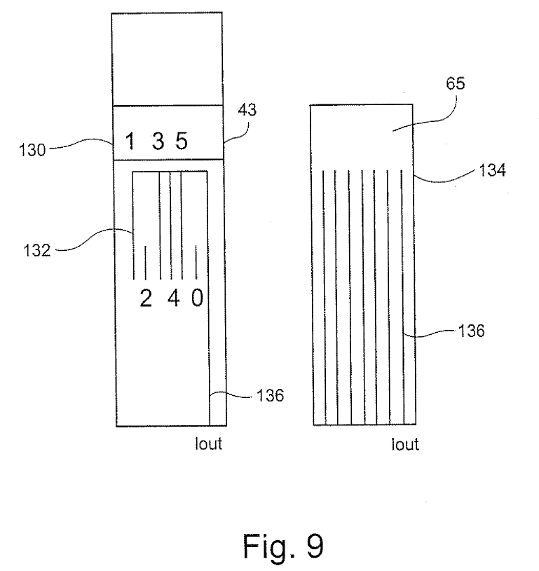

FIG. 9 is a front view of electrical contact pads 43, 65 for the cartridge and device. The contact pad 43 for the cartridge may be mounted on an outer surfaces of one or both of the prongs 62. The contact pad 65 for the device may be mounted on an inside surface 63 of the receiver at the distal end of the device (See FIG. 6).

The contact pad 43 on the prong may be an encoded series of elecro-plated contacts 130 that identifies the cartridge head. For example, the coding may be vertical conductive lines 132 on the pad and arranged in parallel. The lines 132 may each be associated with a particular reference number, e.g., 1 to 5. By encoding the contact 130 with select lines, e.g., 1, 3 and 5, the contact is coded with a prescribed reference indicator. The reference indicator identifies the cartridge and distinguishes the cartridge from other cartridges. The opposing contact pad 65 on the receiver of the device is a generic contact that establishes electrical contact with all of the possible selected lines on the contact pad 43 on the cartridge.

A microcontroller or other control logic in the electronics of the printed circuit board 25 of the device is in electrical communication with the contact pad 65 in the receiver. When the contact pad 65, 134 in the receiver electrically contacts the contact pad 43, 130 of the cartridge, the microcontroller or control logic detects the presence of the selected lines 132 of the contact pad 43 on the cartridge. Based on the detection of the selected lines 132 on pad 34 of the cartridge head, the controller can determine if the cartridge is suitable for the device, whether the cartridge has been previously used in the device and/or the appropriate current levels and current application period for the cartridge. The microcontroller or control logic may store in memory the codes, as detected from the contact pads, of each cartridge used by the device. If the microcontroller or control logic cartridge determines that a newly inserted cartridge has been previously used by the device (by determining that the selected lines on a contact pad of the cartridge are the same as a selected lines of a cartridge previously used in the device), the microcontroller or control logic may disable the device and prevent the application of current to the active electrode in the cartridge.

A conductive path extending on the cartridge between the contact pad 43 on the prongs and a lid with a tab to break the conductive path are optional if the cartridge has a contact pad 43 that is encoded. If the cartridge does not have a conductive path, the cartridge may have a single encoded contact pad 43 on one prong that provides an electrical contact between the power source of the device and the active electrode in the cartridge, and is readable with information regarding the cartridge.

Further, the opposing contact pads 43, 65, 130, 134 both include a line-out (lout) power lines 136 that provide an electrical path for current from the power source in the device to the active electrode in the cartridge. The use of the lout lines reduces the risk that a user will be burned by inadvertently applying current to his skin using the device without a cartridge because the lout line is internal to the receive in the device and not easily put in contact with skin. The lout lines 136 also avoid the need for a pin and interlock switch.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. An applicator cartridge for use with a device for electrokinetically delivering a medicament to a treatment site, the cartridge comprising:
an applicator head including an active electrode, and a conductive path extending between a pair of electrical contacts, wherein the electrical contacts electrically engage respective contacts on the device when the cartridge is inserted into the device;
a matrix support surface of said applicator head adjacent the active electrode;
a matrix attached to the matrix support surface and in contact with the active electrode, the matrix having an exposed surface adapted to be applied to the treatment site;
a medicament or a medicament and an electrically conductive carrier carried by said matrix;
a removable lid covering the exposed surface of the matrix and sealed to the applicator head, the lid is coupled to the applicator head, wherein removal of the lid breaks the conductive path.

2. The applicator as in claim 1 wherein the lid includes a tab of the lid sealed to the conductive path, as the lid peels off the applicator head the removal of the tab breaks the conductive path.

3. The applicator as in claims 1 wherein the matrix is attached in a recess of the head, the head includes a rim at the periphery of the recess, and the lid is sealed to the rim.

4. The applicator as in claim 1 wherein the lid includes a tab sealed to a side surface of the rim and the conductive path includes conductive strips on the side surface, wherein the tab is secured to the conductive strips while the lid is sealed to the applicator head.

5. The applicator as in claim 1 wherein the lid includes a conductive tab that adheres to the side surface and forms part of the conductive path while the lid is secured to the cartridge.

6. The applicator as in claim 1 wherein said lid comprises layers including a metallic material, and the metallic material layer includes tab is electrically connected to the conductive path.

7. The applicator as in claim 1 wherein said lid is heat sealed to the applicator head.

8. The applicator as in claim 1 wherein the contacts on the applicator head are metallic strips adhering to the applicator head on at least one side of prongs extending from the lid.

9. An applicator cartridge for use with a device for electrokinetically delivering a medicament to a treatment site comprising:
an applicator head including an active electrode and a conductive path extending between contacts of the head wherein the contacts electrically contact the device when the cartridge is inserted in the device;
a matrix support surface of said applicator head adjacent the active electrode;
a matrix attached to the matrix support surface and in contact with the active electrode, the matrix having an exposed surface adapted to be applied to the treatment site;
a medicament or a medicament and an electrically conductive carrier carried by said matrix;
a removable lid covering the exposed surface of the matrix and sealed to the applicator head, wherein the lid is coupled to the applicator head and completes the conductive path of the applicator head, wherein removal of the lid breaks the conductive path.

10. The applicator as in claim 9 where the lid includes a tab sealed to the applicator head and engaging the conductive path extending between contacts on the applicator head, wherein removal of the lid breaks the conductive path.

11. The applicator as in claim 10 wherein the tab is fixed to the conductive path and the tab is releasably attached to the applicator head.

12. The application as in claim 9 wherein the conductive path is a metallic foil strip.

13. A method of preventing reuse of an applicator cartridge for use with a device for electrokinetically delivering medicament to a treatment site on skin, the method comprising:
mounting a medicament pad in the applicator cartridge wherein the medicament pad is in electrical contact with an active electrode and has an exposed front surface to be applied to the treatment site;
sealing a lid to the applicator cartridge to cover the front surface of the medicament pad;
establishing a conductive path on the applicator cartridge that extends between contacts on the cartridge;
inserting the applicator cartridge in the device and making electrical connections between the device and the cartridge;
applying an electrical current from the device through the conductive path on the cartridge;
if the electrical current passes through the conductive path, authorizing the applicator cartridge to deliver the medicament to the treatment site;
removing the lid from the applicator cartridge;
applying the exposed front surface to the treatment site and electrokinetically delivering the medicament through the treatment site, and
by the removal of the lid, breaking the conductive path and disabling the device from delivering the medicament to the treatment site if the cartridge is reinserted into the device.

14. A method as in claim 13 wherein the authorization of the applicator cartridge is for a single delivery of medicament to the treatment site.

15. A method as in claim 13 wherein the authorization of the applicator cartridge is for a predetermined period of time.

16. A method as in claims 13 includes determining whether the current passes through the conductive path within a predetermined period of time after the cartridge is inserted into the device.

17. A method as in claim 13 including determining whether the current passes through the conductive path within three seconds after the cartridge is inserted into the device.

18. A method as in claim 13 wherein removal of the lid creates a non-conductive gap in the conductive path.

19. A method as in claim 13 wherein the lid includes a tab sealed to the conductive path, and removal of the lid includes removal of the tab to break the conductive path.

20. A method as in claims 13 wherein insertion of the cartridge into the device automatically triggers the device to apply an electrical potential across the contacts to pass current through the conductive path.

21. A method as in claim 19 wherein the conductive path extends between prongs of the applicator cartridge and the device applies an electric potential to contacts on the prongs.

22. A method of preventing reuse of an applicator cartridge for use with a device for electrokinetically delivering medicament to a treatment site on skin, the method comprising:
mounting a medicament pad in the applicator cartridge wherein the medicament pad is in electrical contact with an active electrode and has an exposed front surface to be applied to the treatment site;
including an electronically readable indicia on the applicator cartridge, wherein the indicia identifies the cartridge;
inserting the applicator cartridge in the device and making electrical connections between the device and the active electrode and with the indicia;
electronically reading the indicia with a processor or logic circuit in the device to determine if the cartridge has been previously used to deliver a medicament;
applying the exposed front surface to the treatment site;
if the determination is that the cartridge has not been previously used, applying electrical current from the device to the active electrode on the cartridge, and
by the application of the electrical current to the active electrode, electrokinetically delivering the medicament to the treatment site.

23. The method of claim 22 further comprising preventing the application of electrical current from the active electrode of the device upon a determination that the cartridge has been used previously.
